(19) United States
(12) Patent Application Publication
(10) Pub. No.: US 2014/0142042 A1
(43) Pub. Date: May 22, 2014

(54) SKIN WOUND HEALING AND SCAR REDUCTION WITH PROSTAGLANDIN EP4 AGONIST COMBINATIONS

(71) Applicant: Allergan, Inc., Irvine, CA (US)

(72) Inventors: Guang L. Jiang, Irvine, CA (US); Robert M. Burk, Laguna Beach, CA (US); Wha-Bin Im, Irvine, CA (US); Larry A. Wheeler, Irvine, CA (US)

(73) Assignee: Allergan, Inc., Irvine, CA (US)

(21) Appl. No.: 14/078,823

(22) Filed: Nov. 13, 2013

Related U.S. Application Data
(60) Provisional application No. 61/727,553, filed on Nov. 16, 2012.

(51) Int. Cl. C07D 333/56; C07D 409/12; C07D 265/10; A61K 45/06; A61K 31/4025; A61K 31/381; A61K 31/535; C07D 211/76; A61K 31/45

(52) U.S. Cl. CPC C07D 333/56; C07D 211/76; C07D 409/12; C07D 265/10; A61K 31/45; A61K 31/4025; A61K 31/381; A61K 31/535; A61K 45/06
USPC 514/18.7; 514/327; 514/422; 514/438; 514/228.8; 514/18.6; 514/44 A

(57) ABSTRACT

A combination of a prostaglandin EP4 agonist and an effective amount of: a prostaglandin EP2 agonist, a skin growth factor, a small peptide, a small inhibitory RNA targeting excess chronic inflammation or fibrosis, a cytokine with beneficial anti-inflammatory activity, an adenosine A2a receptor agonist, an anti-oxidant, or a combination thereof, may be used to treat skin wounds or scars.

### CROSS REFERENCE TO RELATED APPLICATIONS

This application claims the benefit of U.S. Provisional Application Ser. No. 61/727,553 filed on Nov. 16, 2012, which is incorporated by reference herein in its entirety.

### BACKGROUND OF THE INVENTION

There is a continuing need for improved methods of treating skin wounds or scars.

### SUMMARY OF THE INVENTION

A combination of a prostaglandin EP4 agonist with another therapeutically active agent may be used to treat skin wounds or scars. Some embodiments include a method of treating a skin wound or a scar comprising administering an effective amount of a prostaglandin EP4 agonist and an effective amount of: a prostaglandin EP2 agonist, a skin growth factor, a small peptide, a small inhibitory RNA targeting excess chronic inflammation or fibrosis, a cytokine with beneficial anti-inflammatory activity, an adenosine A2a receptor agonist, an anti-oxidant, or a combination thereof, to a mammal in need thereof.

Activation of EP2 by agonists increases intracellular cAMP, while activation of EP4 promotes ERK phosphorylation and intracellular cAMP to a minor extent. Enhancement of intracellular cAMP inhibits skin fibrosis which represents TGF- -induced transformation of skin fibroblasts to myofibroblasts, as measured with the expression of -smooth muscle actin. FIG. 1 shows that an EP2 agonist, when applied with TGF-1 in human skin fibroblast cell cultures, blocked -SMA formation more effectively than Compound 2 (EP4).

ERK phosphorylation by EP4 agonists, on the other hand, enhances cellular growth factors, such as bFGF and VEGF, which promote skin angiogenesis (FIGS. 2 and 3). Scar-less skin wound healing would be aided not only by the cAMP-induced inhibition of excessive fibrosis, which is the major contributor to scar formation, and by rapid wound healing from EP4-induced angiogenesis. The combination of EP2 and EP4 agonists, therefore, would be beneficial for rapid scar less skin wound healing.

In some embodiments, a prostaglandin EP4 agonist may be represented by Formula 1:

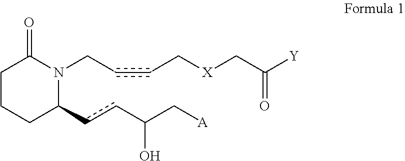

wherein a dashed line indicates the presence or absence of a bond; A is optionally substituted phenyl; X is CH₂, O, or S; Y is OR¹ or NR¹R²; and R¹ and R² are independently H or C₁₋₆ alkyl.

In some embodiments, a prostaglandin EP4 agonist may be represented by Formula 10:

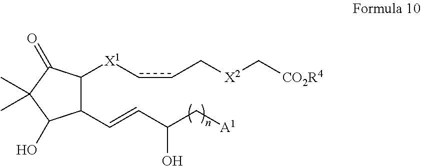

wherein a dashed line indicates the presence or absence of a bond; X¹ and X² are independently S, O, or CH₂; n is 1 or 2; R⁴ is H or C₁₋₆ alkyl; and A¹ is optionally substituted phenyl or optionally substituted benzothienyl.

In some embodiments, a prostaglandin EP2 agonist may be represented by Formula 16:

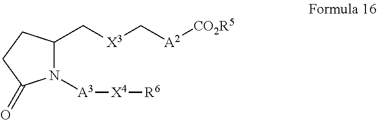

wherein A² may be optionally substituted thien-2,5-yl; A³ may be optionally substituted phenyl; X³ may be CH₂ or O; X⁴ may be C═O or CHOH; R⁵ may be H or C₁₋₆ alkyl; and R⁶ may be C₃₋₈ alkyl.

Some embodiments of the invention include:

1. A method of treating a skin wound or a scar comprising administering an effective amount of a prostaglandin EP4 agonist and an effective amount of: a prostaglandin EP2 agonist, a skin growth factor, a small peptide, a small inhibitory RNA targeting excess chronic inflammation or fibrosis, a cytokine with beneficial anti-inflammatory activity, an adenosine A2a receptor agonist, an anti-oxidant, or a combination thereof, to a mammal in need thereof.
2. The method of embodiment 1, wherein the prostaglandin EP4 agonist is represented by Formula 1:

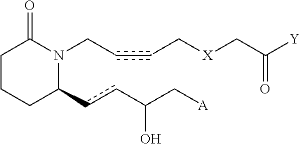

wherein a dashed line indicates the presence or absence of a bond;
A is optionally substituted phenyl;
X is CH₂, O, or S;
Y is OR¹ or NR¹R²;
R¹ and R² are independently H, C₁₋₆ alkyl, hydroxyalkyl, or —CH₂CH₂OH;
and including pharmaceutically acceptable salts, thereof.
   3. The method of embodiments 1 and 2, wherein the prostaglandin EP2 agonist is represented by Formula 16:

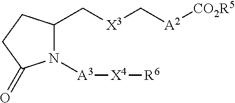

wherein A² is optionally substituted thien-2,5-yl;
A³ is optionally substituted phenyl;
X³ is CH₂ or O;
X⁴ is C═O or CHOH;
R⁵ is H, C₁₋₆ alkyl, hydroxyalkyl, or —CH₂CH₂OH;
R⁶ is C₃₋₈ alkyl;
and including pharmaceutically acceptable salts thereof.
   4. The method of embodiment 1, wherein the prostaglandin EP4 agonist is represented by Formula 10:

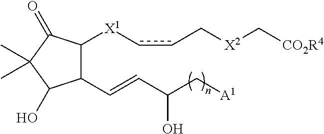

wherein a dashed line indicates the presence or absence of a bond;
X¹ and X² are independently S, O, or CH₂;
n is 1 or 2;
R⁴ is H, C₁₋₆ alkyl, hydroxyalkyl, or —CH₂CH₂OH; and
A¹ is optionally substituted phenyl or optionally substituted benzothienyl; and including pharmaceutically acceptable salts, thereof.
   5. The method of embodiment 1, wherein the prostaglandin EP4 agonist is:

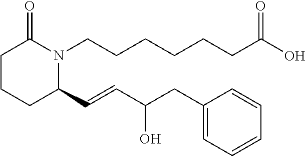 [SMILES: O=C(O)CCCCCCN1C(=O)CCC[C@@H]1/C=C/C(O)Cc1ccccc1]

and including pharmaceutically acceptable salts, thereof.
6. The method of embodiment 3, wherein the prostaglandin EP2 agonist is:

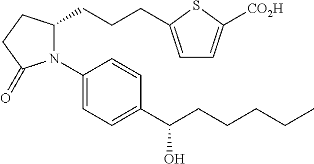 [SMILES: CCCCC[C@H](O)c1ccc(N2C(=O)CC[C@@H]2CCCc2ccc(C(=O)O)s2)cc1]

and including pharmaceutically acceptable salts, thereof.
   7. The method of embodiment 4, wherein the prostaglandin EP4 agonist is:

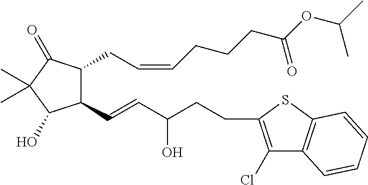 [SMILES: CC(C)OC(=O)CCC/C=C\C[C@H]1C(=O)C(C)(C)[C@@H](O)[C@@H]1/C=C/C(O)CCc1sc2ccccc2c1Cl]

and including pharmaceutically acceptable salts, thereof.
   8. The method of embodiment 4, wherein the prostaglandin EP4 agonist is:

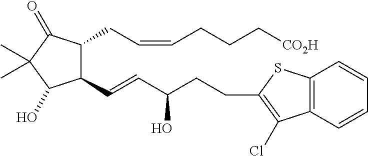 [SMILES: CC1(C)C(=O)[C@H](C/C=C\CCCC(=O)O)[C@@H](/C=C/[C@H](O)CCc2sc3ccccc3c2Cl)[C@@H]1O]

and including pharmaceutically acceptable salts, thereof.
   9. The method of embodiment 1, comprising administering an effective amount of a prostaglandin EP4 agonist and an effective amount of a prostaglandin EP2 agonist.
   10. The method of embodiment 9, wherein the prostaglandin EP4 agonist and the prostaglandin EP2 agonist are administered topically.
   11. The method of embodiment 9, wherein the prostaglandin EP4 agonist and the prostaglandin EP2 agonist are administered orally.
   12. The method of embodiment 9, wherein the prostaglandin EP4 agonist and the prostaglandin EP2 agonist are administered in a single composition.
   13. The method of embodiment 9, wherein the prostaglandin EP4 agonist and the prostaglandin EP2 agonist are administered at least daily for about 1 day to about 30 days.
   14. The method of embodiment 1, wherein the EP4 agonist is

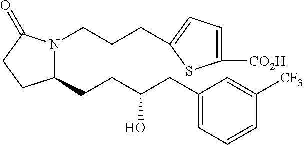 [SMILES: O=C(O)c1ccc(CCCN2C(=O)CC[C@@H]2CC[C@@H](O)Cc2cccc(C(F)(F)F)c2)s1]

including pharmaceutically acceptable salts, thereof.
15. The method of embodiment 1, wherein the EP4 agonist is

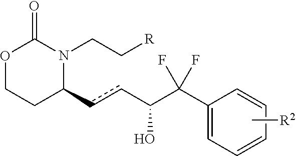

including pharmaceutically acceptable salts thereof.
16. The method of embodiment 1 wherein the EP4 agonist and the additional compound are applied directly to the skin wound or the scar.
17. The method of embodiment 1 wherein the EP4 agonist and the additional compound are applied directly to the skin surrounding wound or the scar.
18. The method of embodiment 1 wherein the EP4 agonist and the additional compound are applied to a surgical site from selected from the group consisting of before, during or after surgery.
19. The method of embodiment 1 wherein the EP4 agonist and the additional compound are applied to a skin wound or scar by injection into the skin wound or scar.
20. The method of embodiment 2 wherein the additional compound is an EP2 agonist.

### BRIEF DESCRIPTION OF DRAWINGS

FIG. 1 shows that an EP2 agonist, when applied with TGF-1 in human skin fibroblast cell cultures, blocked -SMA formation more effectively than Compound 2 (EP4);

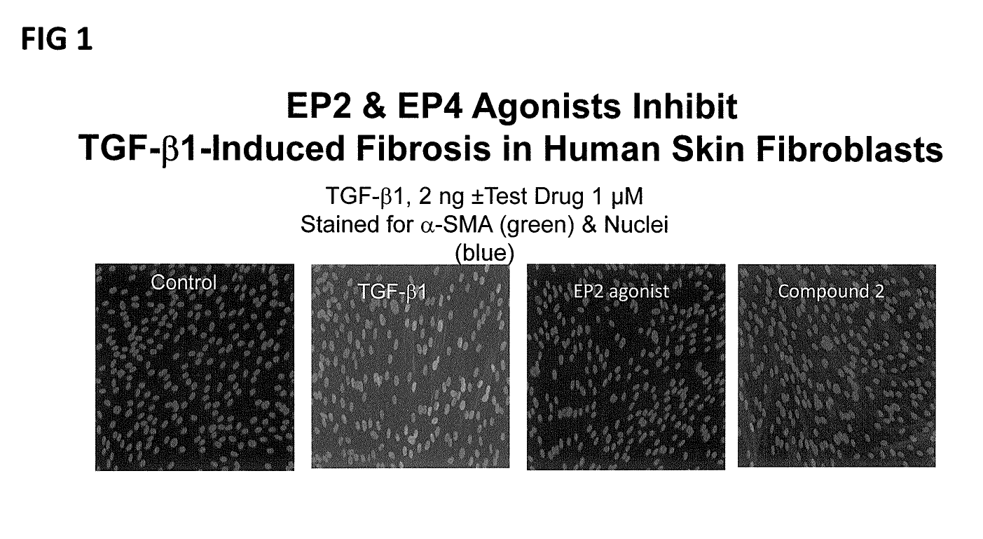

FIG. 2 shows that Compound 2 VEGF expression at incisional wound sites; and

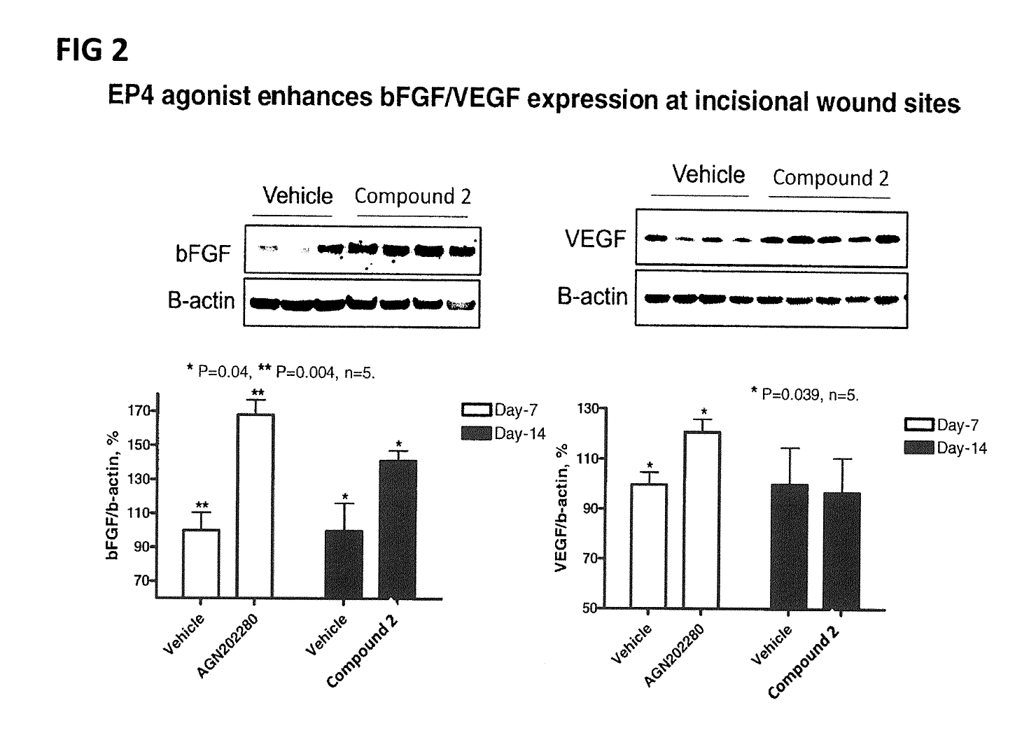

FIG. 3 shows that the EP4 receptor promotes angiogenesis via ERK activation.

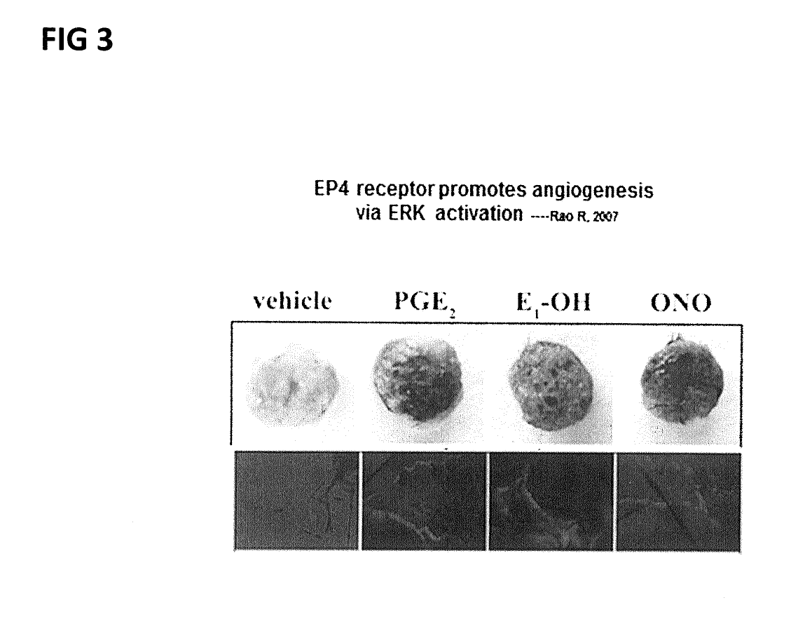

### DETAILED DESCRIPTION OF THE INVENTION

A skin wound or a scar may be treated by administering an effective amount of a prostaglandin EP4 agonist and an effective amount of: a prostaglandin

EP2 agonist, a skin growth factor, a small peptide, a small inhibitory RNA targeting excess chronic inflammation or fibrosis, a cytokine with beneficial anti-inflammatory activity, an adenosine A2a receptor agonist, an anti-oxidant, or a combination thereof, (collectively referred to as “EP4 combinations”) to a mammal in need thereof.

An EP4 combination may be administered topically in a dermatological composition, or in systemic dosage form such as an oral tablet, capsule, pill, etc. Two therapeutically active agents of an EP4 combination, such as a prostaglandin EP4 agonist and a prostaglandin EP2 agonist, may be administered separately or in a single composition. An EP4 combination may be administered at least daily, at least twice daily, at least thrice daily, or more often. An EP4 combination may be administered for at least 1 day, at least 7 days, up to 15 days, up to 30 days, or for longer.

A skin wound includes any wound affecting the skin, such as from cosmetic or other surgical procedures, accidents, and sports-related injuries. A scar includes any discoloration or aberration in skin that remains after a wound has healed.

The terms “treat,” “treatment,” “treating” or related forms include diagnosis, cure, mitigation, treatment, or prevention of disease or injury, such as skin wounds, in man or other animals, or the administration of a composition such as an EP4 combination to affect the structure or any function of the body of man or other animals, such as to reduce scarring.

In some embodiments, a prostaglandin EP4 agonist may be represented by Formula 1:

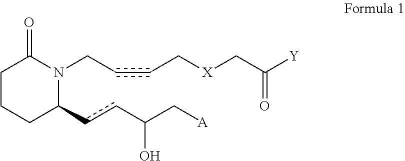

wherein a dashed line indicates the presence or absence of a bond; A is optionally substituted phenyl; X is CH₂, O, or S; Y is OR¹ or NR¹R²; and R¹ and R² are independently H, C₁₋₆ alkyl. hydroxyalkyl, or —CH₂CH₂OH.

The phrase “optionally substituted,” such as “optionally substituted phenyl” includes the unsubstituted moiety, or the moiety having 1 or more substituents. For example, optionally substituted phenyl includes unsubstituted phenyl, and phenyl having 1, 2, 3, 4, or 5 substituents. A substituent may be any atom or group that can replace hydrogen on the phenyl ring. Examples include hydrocarbon groups having from 1 to 12 carbon atoms; heteroatom-containing organic groups such as those comprising hydroxyl, ether, carboxyl, keto, ester, amide, carbamate, urea, thioether, thiol, halo, cyano, and other functional groups; halo such as F, Cl, Br, I; hydroxyl; nitro. In some embodiments, a substituent may have: 1) a molecular weight of about 15 atomic mass units (amu) to about 500 amu, about 15 amu to about 100 amu, and/or about 15 amu to about 50 amu; and/or 2) about 0-12, about 0-6, or about 0-3 carbon atoms, about 0-6 or about 0-3 atoms independently selected from O, N, or S, and/or about 0-24, 0-13, or 1, 2, or 3 halogen atoms. In some embodiments, a substituent may be R^{a}, OR^{a}, COR^{a}, CO₂R^{a}, OCOR^{a}, NR^{a}R^{b}, CONR^{a}R^{b}, F, Cl, I, or CF₃.

The structures of some of the rings or ring systems referred to herein are depicted below. Any of these rings or ring systems may be optionally substituted, where any hydrogen in a ring or a ring system may be replaced by a substituent. Unless attachment is indicated, a ring or a ring system may attach at any position.

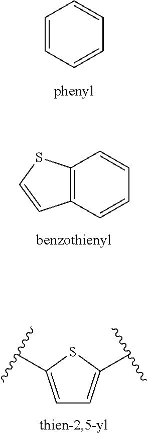 [SMILES: c1ccccc1 ; c1ccc2sccc2c1 ; *c1ccc(*)s1]

Any R^{a} referred to herein may be H, C1-C6 alkyl (such as CH₃, C₂, C₃H₇) or halogen.

Any R^{b} referred to herein may be H, C1-C6 alkyl (such as CH₃, C₂H₅, C₃H₇), or halogen.

The term “molecular weight” may be applied herein to a substituent or any other part of a molecule to indicate the sum of the masses of the individual atoms of a substituent even though a substituent or part or a molecule may not actually be a “molecule.”

As used herein the term “alkyl” has the broadest meaning generally understood in the art, and may include a moiety composed of carbon and hydrogen containing no double or triple bonds. Alkyl may be linear alkyl, branched alkyl, cycloalkyl, or a combination thereof. In some embodiments, alkyl may include C₁₋₆ linear alkyl, such as methyl (—CH₃), ethyl (—CH₂CH₃), n-propyl (—CH₂CH₂CH₃), n-butyl (—CH₂CH₂CH₂CH₃), n-pentyl (—CH₂CH₂CH₂CH₂CH₃), n-hexyl (—CH₂CH₂CH₂CH₂CH₂CH₃), etc.; C₃₋₆ branched alkyl, such as C₃H₇ (e.g. iso-propyl), C₄H₉ (e.g. branched butyl isomers), C₅H₁₁ (e.g. branched pentyl isomers), C₆H₁₃ (e.g. branched hexyl isomers), etc.; C₃₋₆ cycloalkyl, such as cyclic C₃H₅ (e.g. cyclopropyl), cyclic C₄H₇ (e.g. cyclobutyl isomers such as cyclobutyl, methylcyclopropyl, etc.), cyclic C₅H₉ (e.g. cyclopentyl isomers such as cyclopentyl, methylcyclobutyl, dimethylcyclopropyl, etc.) cyclic C₆H₁₁ (e.g. cyclohexyl isomers), etc.; and the like.

With respect to Formula 1 and other structural formulas presented below, any dashed line indicates the presence or absence of a bond. Thus, some compounds may be represented by any of Formulas 2-6.

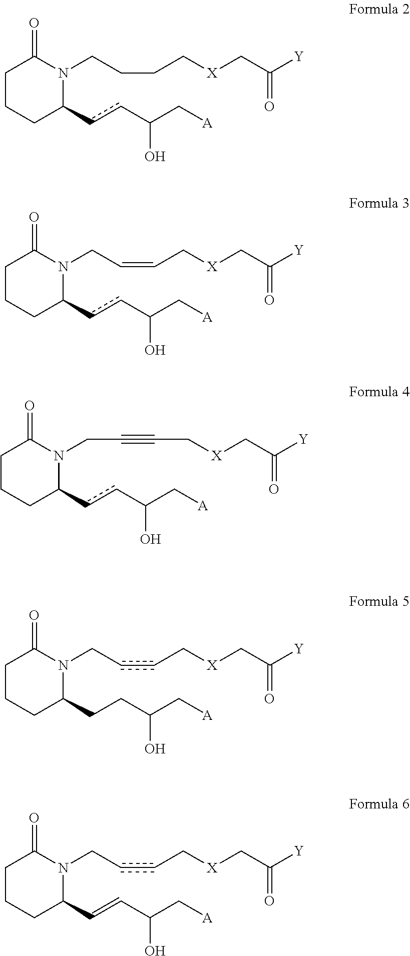

and including pharmaceutically acceptable salts, thereof.

With respect to any relevant formula or structural depiction herein, A may be optionally substituted phenyl. In some embodiments, A may be unsubstituted, or may have 1, 2, or 3 substituents independently selected from R^{a}, OR^{a}, COR^{a}, CO₂R^{a}, OCOR^{a}, NR^{a}R^{b}, CONR^{a}R^{b}, F, Cl, I, or CF₃.

Some embodiments include compounds represented by any of Formulas 7-9:

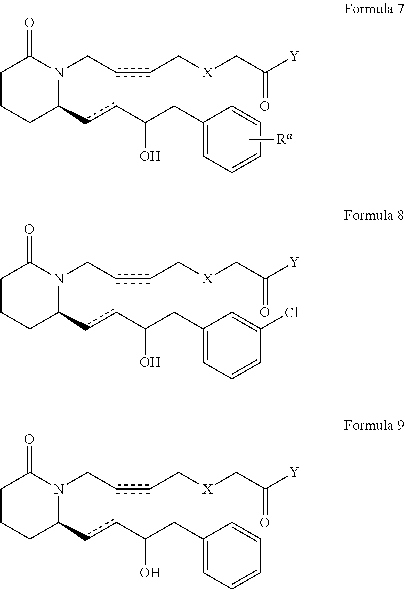

And including pharmaceutically acceptable, thereof.

With respect to any relevant formula or structural depiction herein, such as any of Formulas 1-9, X may be CH₂, O, or S. In some embodiments, X is CH₂.

With respect to any relevant formula or structural depiction herein, such as any of Formulas 1-9, Y may be OR¹ or NR¹R². In some embodiments, Y is OH.

Any R¹ referred to herein may be H, or C₁₋₆ alkyl such as methyl (CH₃), ethyl (e.g. C₂H₅ or CH₂CH₃), propyl isomers (e.g. C₃H₇, including propyl or isopropyl), cyclopropyl (e.g. cyclic C₃H₅), butyl isomers (e.g. C₄H₉), cyclobutyl isomers (e.g. cyclic C₄H₇, including cyclobutyl and methylcyclopropyl), pentyl isomers (e.g. C₅H₁₁), cyclopentyl isomers, hexyl isomers (e.g. C₆H₁₃), cyclohexyl isomers (e.g. cyclic C₆H₁₁), etc.

Any R² referred to herein may be H, or C₁₋₆ alkyl such as methyl (CH₃), ethyl (e.g. C₂H₅ or CH₂CH₃), propyl isomers (e.g. C₃H₇, including propyl or isopropyl), cyclopropyl (e.g. cyclic C₃H₅), butyl isomers (e.g. C₄H₉), cyclobutyl isomers (e.g. cyclic C₄H₇, including cyclobutyl and methylcyclopropyl), pentyl isomers (e.g. C₅H₁₁), cyclopentyl isomers, hexyl isomers (e.g. C₆H₁₃), cyclohexyl isomers (e.g. cyclic C₆H₁₁), etc.

With respect to any relevant formula or structural depiction herein, such as Formula 7, R³ may be R¹, COR², CO₂R¹, OCOR¹, CONR¹R², NR¹COR², OR¹, NR¹R², F, Cl, Br, I, CN, or CF₃.

In some embodiments, a prostaglandin EP4 agonist may be a compound shown below:

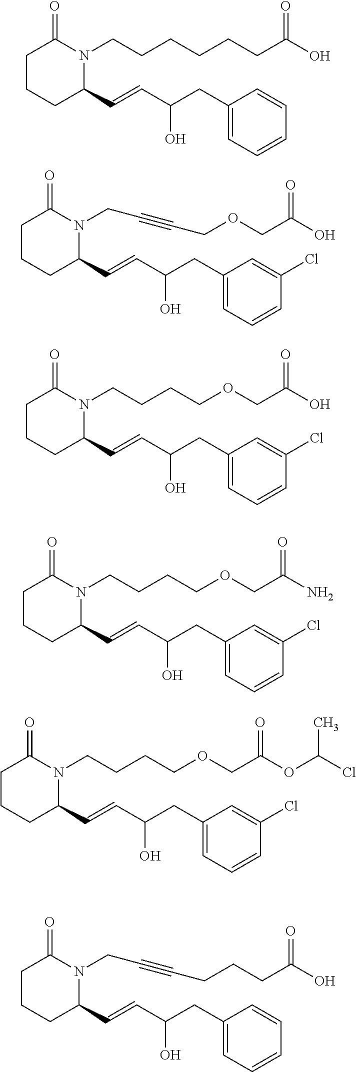

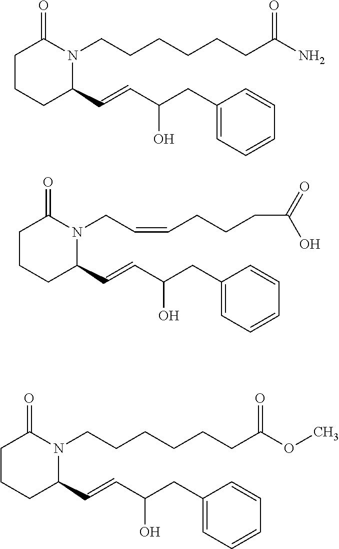 [SMILES: NC(=O)CCCCCCN1C(=O)CCC[C@@H]1/C=C/C(O)Cc1ccccc1 ; O=C(O)CCC/C=C\CN1C(=O)CCC[C@@H]1/C=C/C(O)Cc1ccccc1 ; COC(=O)CCCCCCN1C(=O)CCC[C@@H]1/C=C/C(O)Cc1ccccc1]

and including pharmaceutically acceptable salts, thereof.

These compounds may be prepared as described in U.S. Pat. No. 7,179,820, issued on Feb. 20, 2007 to Old et al, which is incorporated by reference herein in its entirety.

In some embodiments, a prostaglandin EP4 agonist may be represented by Formula 10:

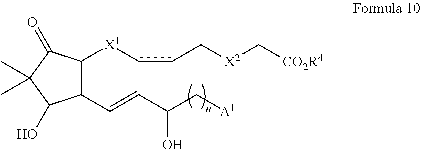

wherein a dashed line indicates the presence or absence of a bond; X¹ and X² are independently S, O, or CH₂; n is 1 or 2; R⁴ is H, C₁₋₆ alkyl hydroxyalkyl, or —CH₂CH₂OH; and A¹ is optionally substituted phenyl or optionally substituted benzothienyl.

Since a dashed line indicates the presence or absence of a bond, some compounds may be represented by Formula 11 or Formula 12:

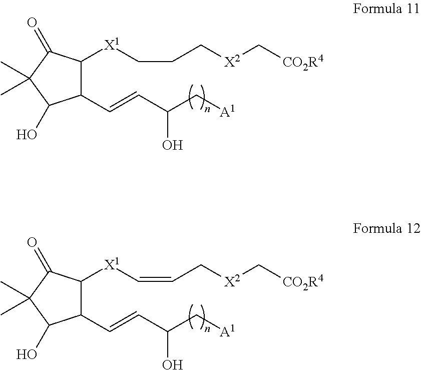

With respect to any relevant formula or structural depiction herein, such as any of Formulas 10-12, A¹ may be optionally substituted phenyl or optionally substituted benzothienyl. In some embodiments, A¹ may be unsubstituted, or may have 1, 2, or 3 substituents independently selected from R^{a}, OR^{a}, COR^{a}, CO₂R^{a}, OCOR^{a}, NR^{a}R^{b}, CONR^{a}R^{b}, F, Cl, I, or CF₃.

With respect to any relevant formula or structural depiction herein, such as any of Formulas 10-12, n may be 1 or 2.

Some embodiments include compounds represented by any of Formulas 13-15:

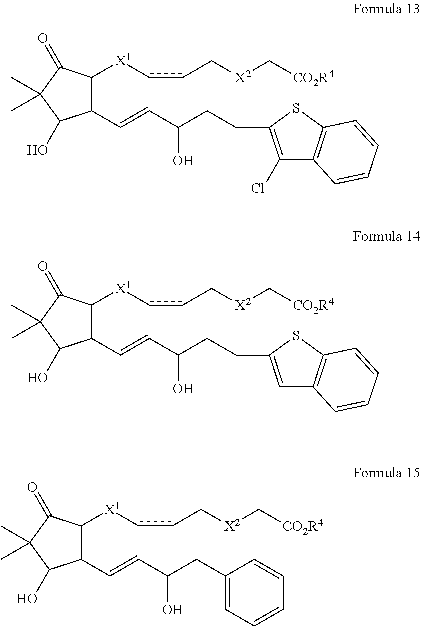

and including pharmaceutically acceptable salts, thereof.

With respect to any relevant formula or structural depiction herein, such as any of Formulas 10-15, X¹ may be S, O, or CH₂.

With respect to any relevant formula or structural depiction herein, such as any of Formulas 10-15, X² may be S, O, or CH₂.

With respect to any relevant formula or structural depiction herein, such as any of Formulas 10-15, R⁴ may be H, or C₁₋₆ alkyl such as methyl (CH₃), ethyl (e.g. C₂H₅ or CH₂CH₃), propyl isomers (e.g. C₃H₇, including propyl or isopropyl), cyclopropyl (e.g. cyclic C₃H₅), butyl isomers (e.g. C₄H₉), cyclobutyl isomers (e.g. cyclic C₄H₇, including cyclobutyl and methylcyclopropyl), pentyl isomers (e.g. C₅H₁₁), cyclopentyl isomers, hexyl isomers (e.g. C₆H₁₃), cyclohexyl isomers (e.g. cyclic C₆H₁₁), etc.

In some embodiments, a prostaglandin EP4 agonist may be a compound and pharmaceutically acceptable salts, thereof, shown below:

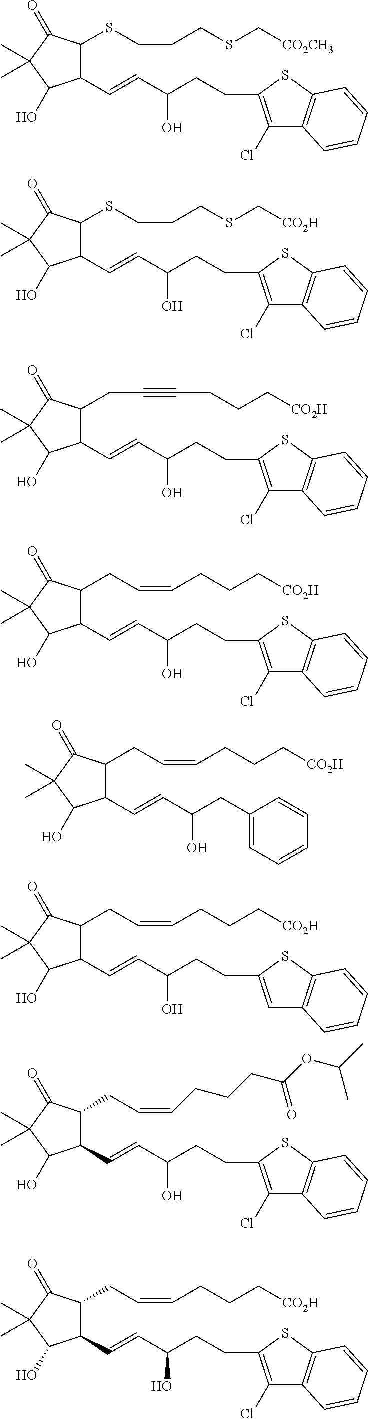

These compounds may be prepared as described in US 20040235958, published on Nov. 25, 2004 by Donde, et. al., which is incorporated by reference herein in its entirety.

For topical compositions, a prostaglandin EP4 agonist may have a concentration in the range from 0.004 to 1%. For oral dosage forms, a prostaglandin EP4 agonist may have a concentration in the range from 0.1 to 10 mg/kg.

In some embodiments, a prostaglandin EP2 agonist may be represented by Formula 16:

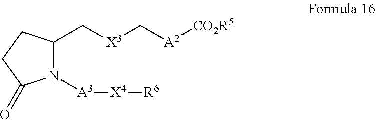

wherein A² may be optionally substituted thien-2,5-yl; A³ may be optionally substituted phenyl; X³ may be CH₂ or O; X⁴ may be C═O or CHOH; R⁵ may be H, C₁-6 alkyl hydroxyalkyl, or —CH₂CH₂OH; and R⁶ may be C₃₋₈ alkyl.

Some prostaglandin EP2 agonists may be represented by one of Formula 17, Formula 18, or Formula 19.

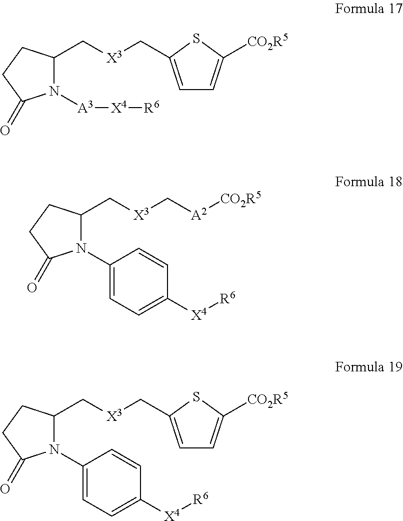

and including pharmaceutically acceptable salts thereof.

With respect to any relevant formula herein, such as Formula 16 or Formula 18, A² may be optionally substituted thien-2,5-yl. In some embodiments, A² may be unsubstituted, or may have 1, 2, or 3 substituents independently selected from R^{a}, OR^{a}, COR^{a}, CO₂R^{a}, OCOR^{a}, NR^{a}R^{b}, CONR^{a}R^{b}, F, Cl, I, or CF₃.

With respect to any relevant formula herein, such as Formula 16 or Formula 17, A³ may be optionally substituted phenyl. In some embodiments, A³ may be unsubstituted, or may have 1, 2, or 3 substituents independently selected from R^{a}, OR^{a}, COR^{a}, CO₂R^{a}, OCOR^{a}, NR^{a}R^{b}, CONR^{a}R^{b}, F, Cl, I, or CF₃.

With respect to any relevant formula herein, such as Formula 16, Formula 17, Formula 18, or Formula 19, R⁵ may be H, or C₁₋₆ alkyl such as methyl (CH₃), ethyl (e.g. C₂H₅ or CH₂CH₃), propyl isomers (e.g. C₃H₇, including propyl or isopropyl), cyclopropyl (e.g. cyclic C₃H₅), butyl isomers (e.g. C₄H₉), cyclobutyl isomers (e.g. cyclic C₄H₇, including cyclobutyl and methylcyclopropyl), pentyl isomers (e.g. C₅H₁₁), cyclopentyl isomers, hexyl isomer (e.g. C₆H₁₃), cyclohexyl isomers (e.g. cyclic C₆H₁₁), etc.

With respect to any relevant formula herein, such as Formula 16, Formula 17, Formula 18, or Formula 19, R⁶ may be Cₘ alkyl, such as propyl isomers (e.g. C₃H₇, including propyl or isopropyl), cyclopropyl (e.g. cyclic C₃H₅), butyl isomers (e.g. C₄H₉), cyclobutyl isomers (e.g. cyclic C₄H₇, including cyclobutyl and methylcyclopropyl), pentyl isomers (e.g. C₅H₁₁), cyclopentyl isomers, hexyl isomer (e.g. C₆H₁₃), cyclohexyl isomers (e.g. cyclic C₆H₁₁), heptyl isomers (e.g. C₇H₁₅), cycloheptyl isomers (e.g. cyclic C₇H₁₃), octyl isomers (e.g. C₈H₁₇), cyclooctyl isomers (e.g. cyclic C₈H₁₅), etc.

With respect to any relevant formula herein, such as Formula 16, Formula 17, Formula 18, or Formula 19, X³ may be CH₂ or O.

With respect to any relevant formula herein, such as Formula 16, Formula 17, Formula 18, or Formula 19, X⁴ may be C═O or CHOH.

In some embodiments, a prostaglandin EP2 agonist may be a compound shown below:

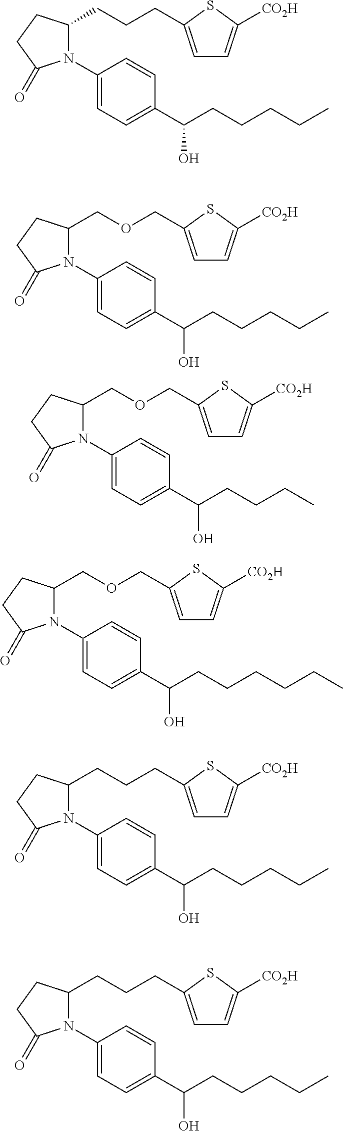

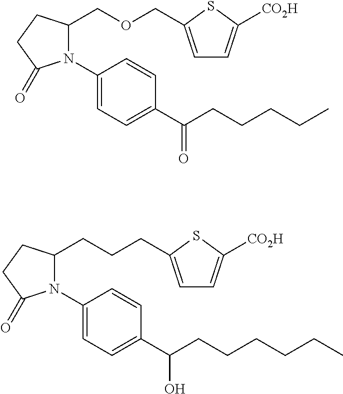 [SMILES: CCCCCC(=O)c1ccc(N2C(=O)CCC2COCc2ccc(C(=O)O)s2)cc1 ; CCCCCCC(O)c1ccc(N2C(=O)CCC2CCCc2ccc(C(=O)O)s2)cc1]

and including pharmaceutically acceptable salts, thereof.

These compounds may be prepared as described in US 20070287742, published on Dec. 13, 2007 by Old et. al., which is incorporated by reference herein in its entirety.

For topical compositions, a prostaglandin EP2 agonist may have a concentration in the range from 0.01% to 1%. For oral dosage forms, a prostaglandin EP2 agonist may have a concentration in the range from 0.1 mg/kg to 20 mg/kg.

In some embodiments, skin wound healing or scar reduction may be promoted with a combination of a prostaglandin EP4 agonist and a skin growth factor. Any skin growth factor may be used including, but not limited to, an epidermal growth factor (EGF), an insulin-like growth factor (IGF), a hepatocyte growth factor (HGF; also known as scatter protein and hepapoietin A;), a vascular endothelial growth factor (VEGF), a platelet-derived growth factor (PDGF), a fibroblast growth factor (FGF), a transforming growth factor beta (TGFβ), a bone morphogenic protein (BMP), or a growth and differentiation factor (GDF).

In some embodiments, an EGF may include a heparin-binding EGF-like growth factor (HB-EGF), a transforming growth factor-α (TGF-α), an amphiregulin (AR), an epiregulin (EPR), an epigen (EPG), a betacellulin (BTC), a neuregulin-1 (NRG1), a neuregulin-2 (NRG2), a neuregulin-3, (NRG3), or a neuregulin-4 (NRG4).

In some embodiments, an IGF may include an IGF-1 or an IGF-2.

In some embodiments, a HGF may include a HGF, a macrophage-stimulating factor (MSP; also known as hepatocyte growth factor-like protein and scatter factor 2), or a livertine.

In some embodiments, a VEGF may include a VEGF-A, a VEGF-B, a VEGF-C, a VEGF-D, or a placenta growth factor (PGF).

In some embodiments, a PDGF may include a PDGFα, a PDGFβ, PDGFγ, or a PDGFδ.

In some embodiments, a FGF may include a FGF1, a FGF2, a FGF3, a FGF4, a FGF5, a FGF6, a FGF7 (also known as a keratinocyte growth factor (KGF)), a FGF8, a FGF9, a FGF10, a FGF16, a FGF17, a FGF18, a FGF19, a FGF20, a FGF21, or a FGF23.

In some embodiments, a TGFβ may include a TGFβ1, a TGFβ2, a TGFβ3, or a TGFβ4.

In some embodiments, a BMP may include a BMP2, a BMP3, a BMP4, a BMP5, a BMP6, a BMP7, a BMP8, or a BMP10.

In some embodiments, a GDF may include a GDF1, a GDF2, a GDF3, a GDF5, a GDF6, a GDF7, a GDF8, a GDF10, a GDF11, or a GDF15.

For topical compositions, a skin growth factor agonist may have a concentration in the range of 1 to 1000 times of their physiological concentrations.

In some embodiments, wound healing or scar reduction may be promoted with a combination of a prostaglandin EP4 agonist and a small peptide. Any small peptides well known to those skilled in the art are contemplated for use in the practice of the invention.

In some embodiments, wound healing or scar reduction may be promoted with a combination of a prostaglandin EP4 agonist and a small inhibitory RNA targeting excess chronic inflammation or fibrosis. Any small inhibitory RNA targeting excess chronic inflammation or fibrosis may be used including, but not limited to, siRNAs against TGF-b1/2, and inflammatory cytokines such as Tumor necrosis factor-alpha.

For topical compositions, an RNA may have a concentration of 100 to 10000 times of physiological concentration of target mRNAs.

In some embodiments, wound healing or scar reduction may be promoted with a combination of a prostaglandin EP4 agonist and a cytokine with beneficial anti-inflammatory activity. Any cytokine with beneficial anti-inflammatory activity may be used including, but not limited to, IL-4, IL-10, IL-13, and the like.

For topical compositions, a cytokine may have a concentration of 100 to 1000 times of physiological concentrations of target mRNAs. For oral dosage forms, a cytokine may have a concentration of 1000 to 10000 times of physiological concentrations.

In some embodiments, wound healing or scar reduction may be promoted with a combination of a prostaglandin EP4 agonist and an adenosine A2a receptor agonist. Any adenosine A2a receptor agonist may be used including, but not limited to, CGS-21680, YT-146, DMPA, Regadenoson, and the like.

For topical compositions, an adenosine A2a receptor agonist may have a concentration of 0.001 to 1%. For oral dosage forms, an adenosine A2a receptor agonist may have a concentration of 1 to 1000 mg/kg.

In some embodiments, wound healing or scar reduction may be promoted with a combination of a prostaglandin EP4 agonist and an anti-oxidant. Any anti-oxidant may be used including, but not limited to, glutathione, vitamin C, vitamin E, and the like.

For topical compositions, an anti-oxidant may have a concentration of 10 to 100 mg. For oral dosage forms, an anti-oxidant may have a concentration of 10 to 10000 mg.

Unless otherwise indicated, any reference to a compound herein by structure, name, or any other means, includes pharmaceutically acceptable salts which are also within the scope of this invention. Reference to a compound is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds may be formed, for example, by reacting a compound with an amount of acid or base, such as an equivalent amount, in a medium such as one in which the salt precipitates or in an aqueous medium followed by lyophilization. Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

Compounds also include prodrugs, such as ester prodrugs; alternate solid forms, such as polymorphs, solvates, hydrates, etc.; tautomers; or any other chemical composition or species that may rapidly convert to a compound described herein under conditions in which a compounds is used as described herein.

Unless stereochemistry is unambiguously depicted, any structure or name for a compound may refer to any stereoisomer or any mixture of stereoisomers.

EP4 combinations may be formulated into a dermatological composition. Some dermatological compositions may comprise a semi-solid or gel-like vehicle that may include a polymer thickener, water, preservatives, active surfactants or emulsifiers, antioxidants, sunscreens, and a solvent or mixed solvent system.

Any polymer thickeners suitable for dermatological application may be used, such as hydrophilic gelling agents frequently used in the cosmetic and pharmaceutical industries. For example, a hydrophilic gelling agent may comprise “CARBOPOL®” (B.F. Goodrich, Cleveland, Ohio), “HYPAN®” (Kingston Technologies, Dayton, N.J.), “NATROSOL®” (Aqualon, Wilmington, Del.), “KLUCEL®” (Aqualon, Wilmington, Del.), or “STABILEZE®” (ISP Technologies, Wayne, N.J.). Any effective amount of gelling agent may be used, such as about 0.2% to about 4% by weight of the composition. A useful weight percent range for “CARBOPOL®” may be about 0.5% to about 2%, a useful weight percent range for “NATROSOL®” and “KLUCEL®” may be about 0.5% to about 4%, and a useful weight percent range for “HYPAN®” or “STABILEZE®” may be about 0.5% to about 4%.

“CARBOPOL®” is one of numerous cross-linked acrylic acid polymers that are given the general adopted name carbomer. These polymers may dissolve in water and may form a clear or slightly hazy gel upon neutralization with a base such as sodium hydroxide, potassium hydroxide, triethanolamine, or other amine bases. “KLUCEL®” is a cellulose polymer that may be dispersed in water and may form a uniform gel upon complete hydration. Other useful gelling polymers may include hydroxyethylcellulose, hydroxypropylcellulose, cellulose gum, MVA/MA copolymers, MVE/MA decadiene crosspolymer, PVM/MA copolymer, etc.

Preservatives may also be used in this dermatological composition and may comprise about 0.05% to 0.5% by weight of the total composition. The use of preservatives may help to reduce or prevent microorganism growth. Some useful preservatives may include methylparaben, propylparaben, butylparaben, chloroxylenol, sodium benzoate, DMDM Hydantoin, 3-Iodo-2-Propylbutyl carbamate, potassium sorbate, chlorhexidine digluconate, etc.

An EP4 combination may be applied in a topical cream or lotion. Topical creams or lotions may be oil-in-water emulsions or water-in-oil emulsions. An oil phase may include but is not limited to fatty alcohols, acids, or esters such as cetyl palmitate, cetyl alcohol, stearyl alcohol, stearic acid, isopropyl stearate, glycerol stearate, mineral oil, white petrolatum, or other oils alone or in combination.

Emulsifiers that may be added to a dermatological composition include, but are not limited to, steareth 20, ceteth 20, sorbitan sesquioleate, sorbitan mono-oleate, propylene glycol stearate, dosium lauroyl sarcosinate, polysorbate 60, or a combination thereof. Preservatives, antioxidants, fragrances, colorants, thickeners, and other additives required to achieve a pharmaceutically or cosmetically acceptable or preferred product may also be included. However, dermatological compositions are not limited to these components since one skilled in the art may be aware of additional components useful in the formulation of topical creams and lotions.

In addition to dermatological compositions, an EP4 combination may be administered systemically as a powder, pill, tablet or the like, or as a solution, emulsion, suspension, aerosol, syrup or elixir suitable for oral or parenteral administration or inhalation.

For solid dosage forms or medicaments, non-toxic solid carriers include, but are not limited to, pharmaceutical grades of mannitol, lactose, starch, magnesium stearate, sodium saccharin, the polyalkylene glycols, talcum, cellulose, glucose, sucrose and magnesium carbonate. The solid dosage forms may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in U.S. Pat. No. 4,256,108, U.S. Pat. No. 4,166,452, and U.S. Pat. No. 4,265,874 to form osmotic therapeutic tablets for control release.

Liquid pharmaceutically administrable dosage forms can, for example, comprise a solution or suspension of one or more of the presently useful compounds and optional pharmaceutical adjutants in a carrier, such as for example, water, saline, aqueous dextrose, glycerol, ethanol and the like, to thereby form a solution or suspension. If desired, a pharmaceutical composition to be administered may also contain minor amounts of nontoxic auxiliary substances such as wetting or emulsifying agents, pH buffering agents and the like. Typical examples of such auxiliary agents are sodium acetate, sorbitan monolaurate, triethanolamine, sodium acetate, triethanolamine oleate, etc. Actual methods of preparing such dosage forms are known, or will be apparent, to those skilled in this art; for example, see Remington's Pharmaceutical Sciences, Mack Publishing Company, Easton, Pa., 16th Edition, 1980. The composition of the formulation to be administered, in any event, may contains a quantity of one or more compounds of an EP4 combination in an amount effective to provide the desired therapeutic effect.

Parenteral administration is generally characterized by injection, either subcutaneously, intramuscularly or intravenously. Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol and the like. In addition, if desired, the injectable pharmaceutical compositions to be administered may also contain minor amounts of non-toxic auxiliary substances such as wetting or emulsifying agents, pH buffering agents and the like.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

The terms “a,” “an,” “the” and similar referents used in the context of describing the invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of any claim. No language in the specification should be construed as indicating any non-claimed element essential to the practice of the invention.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, the claims include all modifications and equivalents of the subject matter recited in the claims as permitted by applicable law. Moreover, any combination of the above-described elements in all possible variations thereof is contemplated unless otherwise indicated herein or otherwise clearly contradicted by context.

In closing, it is to be understood that the embodiments disclosed herein are illustrative of the principles of the claims. Other modifications that may be employed are within the scope of the claims. Thus, by way of example, but not of limitation, alternative embodiments may be utilized in accordance with the teachings herein. Accordingly, the claims are not limited to embodiments precisely as shown and described.

### EXAMPLES

TABLE 1

| | EP₂ | EP₂ | EP₄ | EP₄ |
|---|---|---|---|---|
| | cAMP | Kᵢ | cAMP | Kᵢ |
| Compound | EC₅₀ (nM) | IC₅₀ (nM) | EC₅₀ (nM) | IC₅₀ (nM) |
| 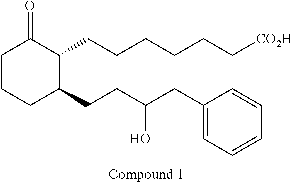 [SMILES: O=C(O)CCCCCC[C@H]1C(=O)CCC[C@@H]1CCC(O)Cc1ccccc1] | >10⁴ | >10⁴ | 0.9 | 81 |
| 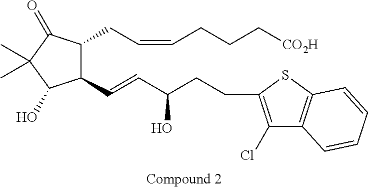 [SMILES: CC1(C)C(=O)[C@H](C/C=C\CCCC(=O)O)[C@@H](/C=C/[C@H](O)CCc2sc3ccccc3c2Cl)[C@@H]1O] | >10⁴ | >10⁴ | 0.3 | 7 |
| 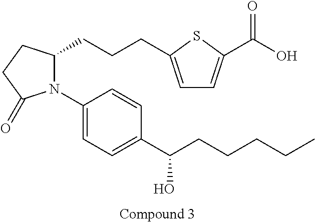 [SMILES: CCCCC[C@H](O)c1ccc(N2C(=O)CC[C@@H]2CCCc2ccc(C(=O)O)s2)cc1] | 0.19 | 21 | >10⁴ | >10⁴ |
| 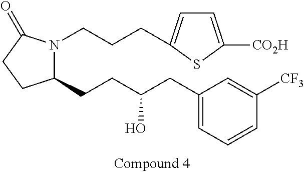 [SMILES: O=C(O)c1ccc(CCCN2C(=O)CC[C@@H]2CC[C@@H](O)Cc2cccc(C(F)(F)F)c2)s1] | 71 | 4647 | 0.08 | 2 |

### Incisional Skin Wound Model and Assessment

Sprague-Dawley rats at 180-200 gram were anesthetized with isoflourane. After shaving, 2-cm long incisions were made on the left and right side of the back, reaching the deep fascia on the back skin of rats under sterile conditions. Incisional wounds were immediately closed with 4.0 sutures, and then topically treated with a vehicle or test drugs at 0.004% twice daily for 5 days. The vehicle used here contains ethanol 30%, propylene glycol 12%, dipropylene glycol 5%, benzyl alcohol 5%, glycerol 3% and normal saline 45%.

Wounds were photographed on day 7. All photos were coded and scored by lay people. Evaluation of wound sites was based on scar width, palpability (elevation) of wound areas, and general progress in healing, using a scale of 0 to 6; the severer a scar, the higher the score. Each scar was divided into 4 regions, separated by suture sites; each quarter was scored independently; the mean of the 4 part scores was recorded as the overall scar score of each incision site.

TABLE 2
Comparison of scar scores of incisional wounds on Day 7 in rats
topically treated with test drugs at 0.004% or vehicle for 5 days.

| | Compound 2 | | Compound 4 | |
|---|---|---|---|---|
| | Vehicle- | Drug- | Vehicle- | Drug- |
| | treated | treated | treated | treated |
| Scar Scores | 1.5 ± 0.3 | 0.60 ± 0.1 | 1.3 ± 0.07 | 0.76 ± 0.07 |

What is claimed is:

1. A method of treating a skin wound or a scar comprising administering an effective amount of a prostaglandin EP4 agonist and an effective amount of an additional compound selected from the group consisting of a prostaglandin EP2 agonist, a skin growth factor, a small peptide, a small inhibitory RNA targeting excess chronic inflammation or fibrosis, a cytokine with beneficial anti-inflammatory activity, an adenosine A2a receptor agonist, an anti-oxidant, or a combination thereof, to a mammal in need thereof.

2. The method of claim 1, wherein the prostaglandin EP4 agonist is represented by Formula 1: 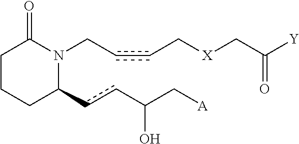
wherein a dashed line indicates the presence or absence of a bond;
A is optionally substituted phenyl;
X is CH₂, O, or S;
Y is OR¹ or NR¹R²;
R¹ and R² are independently H, C₁₋₆ alkyl, hydroxyalkyl, or —CH₂CH₂OH; and including pharmaceutically acceptable salts, thereof.

3. The method of claim 1, wherein the prostaglandin EP2 agonist is represented by Formula 16: 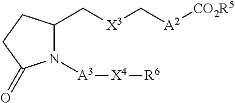
wherein A² is optionally substituted thien-2,5-yl;
A³ is optionally substituted phenyl;
X³ is CH₂ or O;
X⁴ is C═O or CHOH;
R⁵ is H, C₁₋₆ alkyl, hydroxyalkyl, or —CH₂CH₂OH;
R⁶ is C₃₋₈ alkyl;
and including pharmaceutically acceptable salts, thereof.

4. The method of claim 1, wherein the prostaglandin EP4 agonist is represented by Formula 10: 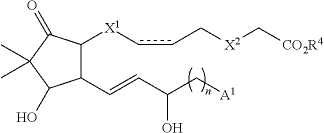
wherein a dashed line indicates the presence or absence of a bond;
X¹ and X² are independently S, O, or CH₂;
n is 1 or 2;
R⁴ is H, C₁₋₆ alkyl, hydroxyalkyl, or —CH₂CH₂OH; and
A¹ is optionally substituted phenyl or optionally substituted benzothienyl; and including pharmaceutically acceptable salts, thereof.

5. The method of claim 1, wherein the prostaglandin EP4 agonist is: 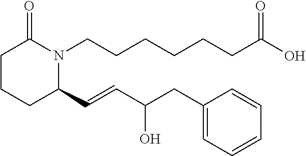 [SMILES: O=C(O)CCCCCCN1C(=O)CCC[C@@H]1/C=C/C(O)Cc1ccccc1]
and including pharmaceutically acceptable salts, thereof.

6. The method of claim 3, wherein the prostaglandin EP2 agonist is: 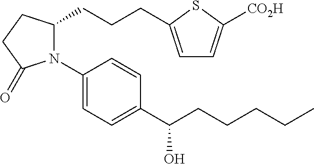 [SMILES: CCCCC[C@H](O)c1ccc(N2C(=O)CC[C@@H]2CCCc2ccc(C(=O)O)s2)cc1]
and pharmaceutically acceptable salts, thereof.

7. The method of claim 4, wherein the prostaglandin EP4 agonist is: 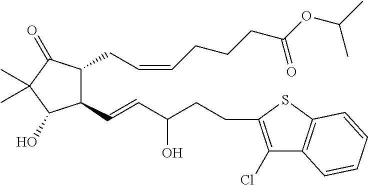 [SMILES: CC(C)OC(=O)CCC/C=C\C[C@H]1C(=O)C(C)(C)[C@@H](O)[C@@H]1/C=C/C(O)CCc1sc2ccccc2c1Cl]
and pharmaceutically acceptable salts, thereof.

8. The method of claim 4, wherein the prostaglandin EP4 agonist is: 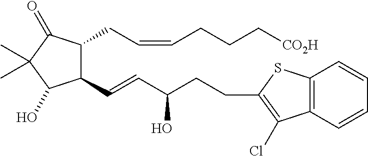 [SMILES: CC1(C)C(=O)[C@H](C/C=C\CCCC(=O)O)[C@@H](/C=C/[C@H](O)CCc2sc3ccccc3c2Cl)[C@@H]1O]
and pharmaceutically acceptable salts, thereof.

9. The method of claim 1, comprising administering an effective amount of a prostaglandin EP4 agonist and an effective amount of a prostaglandin EP2 agonist.

10. The method of claim 9, wherein the prostaglandin EP4 agonist and the prostaglandin EP2 agonist are administered topically.

11. The method of claim 9, wherein the prostaglandin EP4 agonist and the prostaglandin EP2 agonist are administered orally.

12. The method of claim 9, wherein the prostaglandin EP4 agonist and the prostaglandin EP2 agonist are administered in a single composition.

13. The method of claim 9, wherein the prostaglandin EP4 agonist and the prostaglandin EP2 agonist are administered at least daily for about 1 day to about 30 days.

14. The method of claim 1, wherein the EP4 agonist is 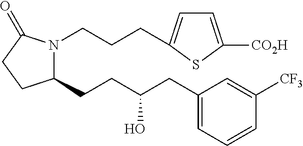 [SMILES: O=C(O)c1ccc(CCCN2C(=O)CC[C@@H]2CC[C@@H](O)Cc2cccc(C(F)(F)F)c2)s1]
and pharmaceutically acceptable salts, thereof.

15. The method of claim 1, wherein the EP4 agonist is 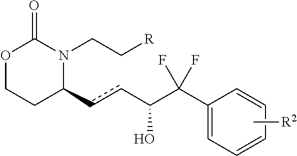
and pharmaceutically acceptable salts, thereof.

16. The method of claim 1 wherein the EP4 agonist and the additional compound are applied directly to the skin wound or the scar.

17. The method of claim 1 wherein the EP4 agonist and the additional compound are applied directly to the skin surrounding the skin wound or the scar.

18. The method of claim 1 wherein the EP4 agonist and the additional compound are applied to a surgical site from selected from the group consisting of before, during or after surgery.

19. The method of claim 1 wherein the EP4 agonist and the additional compound are applied to a skin wound or scar by injection into the skin wound or scar.

20. The method of claim 2 wherein the additional compound is an EP2 agonist.
